SEQUENCE LISTING (15 sequences)

SEQ ID NO: 1 - protein, 16 aa, synthetic construct
AEAEAKAKAEAEAKAK

SEQ ID NO: 2 - protein, 8 aa, synthetic construct
IEVEIRVK

SEQ ID NO: 3 - protein, 8 aa, synthetic construct
IEIEIRIK

SEQ ID NO: 4 - protein, 8 aa, synthetic construct
IELEIRLK

SEQ ID NO: 5 - protein, 12 aa, synthetic construct
IRVKIEVEIRVK

SEQ ID NO: 6 - protein, 12 aa, synthetic construct
IRIKIEIEIRIK

SEQ ID NO: 7 - protein, 12 aa, synthetic construct
IRVEIRVEIRVE

SEQ ID NO: 8 - protein, 12 aa, synthetic construct
IEVEIEVKIRVK

SEQ ID NO: 9 - protein, 8 aa, synthetic construct
KVRIEVEI

SEQ ID NO: 10 - protein, 8 aa, synthetic construct
KIRIEIEI

SEQ ID NO: 11 - protein, 8 aa, synthetic construct
KLRIELEI

SEQ ID NO: 12 - protein, 12 aa, synthetic construct
KVRIEVEIKVRI

SEQ ID NO: 13 - protein, 12 aa, synthetic construct
KIRIEIEIKIRI

SEQ ID NO: 14 - protein, 12 aa, synthetic construct
EVRIEVRIEVRI

SEQ ID NO: 15 - protein, 12 aa, synthetic construct
KVRIKVEIEVEI

(19) United States
(12) Patent Application Publication
(10) Pub. No.: US 2024/0352093 A1
(43) Pub. Date: Oct. 24, 2024

(54) PEPTIDE AND GELATIN HYBRID HYDROGELS AS ECONOMICAL ALTERNATIVES TO COMMON BASEMENT-MEMBRANE MATRICES

(71) Applicant: AGENCY FOR SCIENCE, TECHNOLOGY AND RESEARCH, Singapore (SG)

(72) Inventors: Lingzhi Ashlynn LEE, Singapore (SG); Yi Yan YANG, Singapore (SG)

(73) Assignee: AGENCY FOR SCIENCE, TECHNOLOGY AND RESEARCH, Singapore (SG)

(21) Appl. No.: 18/684,884

(22) PCT Filed: Aug. 19, 2022
(86) PCT No.: PCT/SG2022/050592

§ 371 Date: Feb. 20, 2024

(30) Foreign Application Priority Data
Aug. 19, 2021 (SG) 10202109074X

(51) Int. Cl. C07K 14/78; C07K 7/06; C07K 7/08; C12N 5/09

(52) U.S. Cl. CPC C07K 14/78; C07K 7/06; C07K 7/08; C12N 5/0693; C12N 2533/54

(57) ABSTRACT

Described herein is a hydrogel comprising a peptide and gelatin. The peptide comprises a sequence having at least 8 amino acids with alternating hydrophobic amino acids (X) and hydrophilic amino acids (Y), wherein each hydrophobic amino acid is independently selected from isoleucine (I), valine (V) and leucine (L), each hydrophilic amino acid is independently selected from arginine (R), lysine (K), glutamic acid (E), and aspartic acid (D), wherein the hydrophilic amino acids are selected such that there is at least one arginine and at least one lysine. A kit and method for preparing the hydrogel is described as well. The hydrogel may be used to culture cells and determine an effect of a compound on a cell cultured in the hydrogel.

The present application claims priority to Singapore patent application number 10202109074X titled “Peptide/Gelatin Hybrid Hydrogels as Economical Alternatives to Common Basement-Membrane Matrices” filed on 19 Aug. 2021 which is incorporated by reference herein in its entirety.

### TECHNICAL FIELD

The present application relates to a hydrogel having a peptide and gelatin and the in-vitro and in-vivo use of the hydrogel.

### BACKGROUND

Hydrogels are swollen 3D viscoelastic polymeric networks that have physical properties similar to the natural body tissue. The first use of hydrogels in biomedical applications dates back to the 1960s and have gained popularity ever since because of their appealing properties such as biocompatibility, excellent water retention capacity and tuneable mechanical strength to suit the tissue of interest. Importantly, the engineering potential of hydrogels continues to expand with the progress made in chemistry and biology.

Hydrogel based material are used to create an environment that can support and promote the expansion of cells for both in vitro 3D tumor spheroids cultures and in vivo tumorgrafts. One of the most widely used material for these purposes is Matrigel™, due to its inherent bioactivity and structural similarity to the extracellular matrix. However, the use of such animal-derived scaffolds is limited by the batch-to-batch inconsistency and the presence of xenogenic impurities and the lack of knowledge of detailed components which continues to be elusive and varying.

The main composition of Matrigel include four key basement-membrane ECM proteins: laminin (˜60%), collagen IV (˜30%), entactin (˜8%) and the heparin sulfate proteoglycan perlecan (˜2-3%). Apart from these, it also contains proteins derived from tumours, including growth factors such as transforming growth factor (TGF) family peptides and fibroblast growth factors, as well enzymes such as matrix metalloproteinases (MMPs). The complexity of the composition is also made worse with more than 14,000 peptides and ˜2,000 protein sequences being uncovered through various proteomics studies, all of which are present in varying levels or in some cases, undetected in different batches of Matrigel. Adding to the challenge, Matrigel cannot be easily manipulated with physical or biochemical handling, making it technically demanding to adjust the matrix to direct intended cell behaviours and attain the desired biological outcomes. The presence of xenogeneic contaminants, including multiple components that are biologically active, causes complexity and requires caution in interpreting cellular activity when cultured on the Matrigel. Furthermore, there have also been reports of Matrigel containing viral contaminants, in particular, lactate dehydrogenase-elevating virus (LDHV). LDHV is a naturally occurring virus in mice and can affect the immune system and tumor behavior when Matrigel is used for experiments in animal models. Despite the drawbacks of using Matrigel, it is still widely used for in vitro and in vivo cell culture, mainly due to its obtainability, simplicity of usage and adaptability to different cell types. Importantly, it may also be due to the deficiency in synthetic alternative with performance that is equal or better than Matrigel as a 3D scaffold for cell culture. Nevertheless, there have been recent reports on materials that are synthetically prepared and can provide a more chemically defined and animal components-free matrix for cells to grow. Moreover, synthetic matrices are far more likely to be consistent between batches and they possess the possibility of being chemically modified to better cater for different cell types and applications.

Synthetic materials that have been used as cell culture scaffolds can be classified according to two main types: polymer-based and peptide-based. Polymers that are used to create 3D scaffolds include poly (ethylene glycol), polycaprolactone (PCL), poly (L-lactide) (PLLA), poly (lactide-co-glycolide) (PLGA) and poly (N-isopropyl acrylamide) polyNIPAM. Although the aforementioned synthetic polymers are biocompatible, stable and non-toxic. they are inherently deficient in biological activity and do not provide a conducive environment for the proliferation of cells within the matrices. To counteract this and promote biological recognition of synthetic scaffolds, modification of such polymers have been conducted through the incorporation of cell adhesive proteins (e.g. collagen and fibrinogen) or peptides, such as Arg-Gly-Asp (RGD). However, cell growth within such polymer-peptide conjugate hydrogels is limited. Hydrogels made from peptides were reported. Amphiphilic peptides such as n-AEAEAKAKAEAEAKAK-c (SEQ ID NO: 1, EAK16) can self-assemble into hydrogels.

### SUMMARY

In a first aspect, there is provided a hydrogel comprising a peptide and gelatin, the peptide comprising a sequence having at least 8 amino acids with alternating hydrophobic amino acids (X) and hydrophilic amino acids (Y), wherein each hydrophobic amino acid is independently selected from isoleucine (I), valine (V) and leucine (L), each hydrophilic amino acid is independently selected from arginine (R), lysine (K), glutamic acid (E), and aspartic acid (D), wherein the hydrophilic amino acids are selected such that there is at least one arginine and at least one lysine.

Preferably, the hydrophilic amino acids are selected such that there are at least two hydrophilic amino acids selected from glutamic acid and aspartic acid.

Preferably, the peptide comprises 8 or 12 amino acids.

In an embodiment, four sequential hydrophilic amino acids (Y₁, Y₂, Y₃, and Y₄) in the sequence are selected such that Y₁ and Y₂ are each independently selected from glutamic acid and aspartic acid, and Y₃ and Y₄ are each independently selected from arginine and lysine. In an embodiment, Y₁ and Y₂ is glutamic acid, Y₃ is arginine, and Y₄ is lysine.

In an embodiment, six sequential hydrophilic amino acids (Y₅, Y₆, Y₁, Y₂, Y₃, and Y₄) in the sequence are selected such that Y₁ and Y₂ are each independently selected from glutamic acid and aspartic acid, and Y₅, Y₆, Y₃ and Y=are each independently selected from arginine and lysine. In an embodiment, Y₁ and Y₂ is glutamic acid, Y₅ and Y₃ is arginine, and Y₆ and Y₄ is lysine.

In an embodiment, the sequence comprises a SEQ ID selected from the group consisting of SEQ ID No: 2, SEQ ID No: 3, SEQ ID No: 4, SEQ ID No: 5, SEQ ID No: 6, SEQ ID NO: 7, SEQ ID NO: 8, SEQ ID NO: 9, SEQ ID NO: 10, SEQ ID NO. 11, SEQ ID NO: 12, SEQ ID NO: 13, SEQ ID NO: 14, SEQ ID NO: 15, and SEQ ID NO: 16. Preferably, the sequence consists essentially of the SEQ ID NO, more preferably the sequence consists of the SEQ ID NO.

Preferably, the peptide is present in 0.1 to 2 weight percent of the hydrogel. Preferably, the peptide is present in 0.2 to 1 weight percent of the hydrogel, more preferably the peptide is present in 0.2 to 0.4 weight percent of the hydrogel.

Preferably, gelatin is present in 1 to 10 weight percent of the hydrogel. More preferably, gelatin is present in 1 to 6 weight percent of the hydrogel, even more preferably the gelatin is present in 2 to 3 weight percent of the hydrogel.

In a second aspect, there is provided a method of preparing a hydrogel, the method comprising mixing a peptide and gelatin under suitable conditions to form the hydrogel, the peptide comprising a sequence having at least 8 amino acids with alternating hydrophobic amino acids and hydrophilic amino acids, wherein each hydrophobic amino acid is independently selected from isoleucine (I), valine (V) and leucine (L), each hydrophilic amino acid is independently selected from arginine (R), lysine (K), glutamic acid (E), and aspartic acid (D), wherein the hydrophilic amino acids are selected such that there is at least one arginine and at least one lysine.

Preferably, the suitable conditions include at least one the following conditions: (a) a salt; and (b) a temperature of 20° C. to 40° C.

In a third aspect, there is provided a kit for preparing a hydrogel, the kit comprising a peptide and gelatin, the peptide comprising a sequence having at least 8 amino acids with alternating hydrophobic amino acids and hydrophilic amino acids, wherein each hydrophobic amino acid is independently selected from isoleucine (I), valine (V) and leucine (L), each hydrophilic amino acid is independently selected from arginine (R), lysine (K), glutamic acid (E), and aspartic acid (D), wherein the hydrophilic amino acids are selected such that there is at least one arginine and at least one lysine.

In a fourth aspect, there is provided a method of culturing cells, the method comprising providing the hydrogel according to the first aspect described above; and incubating a cell and the hydrogel under suitable conditions to culture the cell.

In an embodiment, the cell is a tumor cell, preferably a tumor spheroid.

In an embodiment, the cell is a stem cell.

In a fifth aspect, there is provided a method of determining an effect of one or more compounds on a plurality of cells, the method comprising culturing the plurality of cells according to any one of claims 16 to 17; adding the one or more compounds to the plurality of cells; and determining the effect of the one or more compounds on the plurality of cells.

Advantageously, the hydrogel described herein may be an alternative to animal-derived basement membrane products for cell culture applications, with equivalent performance with regards to cell proliferation, spheroid characteristics, biocompatibility, and in vivo tumor growth to presently available products. The hydrogel provided similarly growth and viability of the cancer cells when they were used as scaffolds for in vitro 3D culture of tumor spheroids with similar hypoxic, proliferative and apoptotic characteristics to presently available products. The hybrid hydrogels described herein may be used for 3D cell culture, stem cell culture, in vitro tumor spheroids generation, and in vivo solid tumor development.

Advantageously, the chemical composition of the hydrogel is simple and well-defined, without xenogenic components and having a low cost of production confers many technical advantages over presently available basement membrane products.

### DESCRIPTION OF FIGURES (FIGS.)

FIG. 1 shows the circular dichroism (CD)spectrum of the peptide amphiphiles IVK8. The measurements were performed at 1.0 mg/mL in water or PBS (pH 7.4) at room temperature (23-24° C.)

FIG. 2A shows the changes in viscosity of IVK8/Gelatin I_0.25%/G_3% hybrid hydrogel as a function of shear rate. FIG. 2B shows the dynamic step strain amplitude test of IVK8/Gelatin I_0.25%/G_3% hybrid hydrogel.

FIG. 3 shows the in vitro degradation profile of hydrogels in PBS buffer containing 0.01 wt % of collagenase at 37° C.

FIG. 4 shows the results of HepG2 tumor spheroids cultured in IVK8/Gelatin hybrid hydrogels. Panels A, B, and C respectively show the of size measurements of the HepG2 tumor spheroids over a 7 day period (panel A), the cell viability of the HepG2 tumor spheroids represented by absorbance at 490 nm (panel B), and microscopy images of HepG2 tumor spheroids cultured in IVK8/Gelatin hybrid hydrogels on Day 7 (panel C)). In panel A, for each day the size of the tumor spheroids is shown in the bar chart from left to right in the following order: Geltrex, I_1.0%, I_0.5%. I_0.67%/G_2%, I_0.5%/G_3%, I_0.33%/G_2%, and I_0.25%/G_3%. Scale bar represents 200 μm.

FIG. 5 shows results of SW480 tumor spheroids cultured in IVK8/Gelatin hybrid hydrogels. Panels A, B and C′ respectively show the size measurements of the SW480 tumor spheroids over a 7 day period (panel A), the cell viability represented by absorbance at 490 nm (panel B), and microscopy images of SW480 tumor spheroids cultured in IVK8/Gelatin hybrid hydrogels on Day 7 (panel C). In panel A. for each day the size of the tumor spheroids in the bar chart is shown from left to right in the following order: Geltrex, I_0.33%/G_2%, I_0.25%/G_3%, I_0.33% and I_0.25%. Scale bar represents 200 μm.

FIG. 6A shows the live/dead staining of SW480 spheroids cultured for 7 days in various hydrogel samples. The zone of viable cells appear as green, while the zone of nonviable cells appear as red. Scale bar represents 200 μm. FIG. 6B shows the detection of hypoxia of SW480 spheroids cultured for 7 days in various hydrogel samples. Image It Hypoxia Reagent is non-fluorescent under non-hypoxic conditions and emits red fluorescence under hypoxic conditions. The core of the spheroids appears red as low oxygen levels is detected, while the periphery is non-hypoxic and does not emit red fluorescence. The cells are counterstained with NucBlue Live Ready Probes Reagent (Hoechst 33342) which emits blue fluorescence. Scale bar represents 200 μm. FIG. 6C shows the size measurements of SW480 tumor spheroids cultured in IVK8/Gelatin hybrid hydrogels on Day 7 and subsequently treated with Doxorubicin for 24h. For each of the control and hydrogel, the results are shown from left to right in the following order: t=0h (before addition of Dox; and after 24h, with no addition of Dox, addition of 0.8 μM Dox, addition of 4 μM Dox, addition of 20 μM Dox and addition of 100 μM Dox. FIG. 6D shows the immunohistochemical staining (Ki67 and Cleaved Caspase 3) and H&E Staining of SW480 spheroids cultured for 7 days in various hydrogel samples. Scale bar represents 100 μm.

FIGS. 7A and 7B respectively show the volume and weight measurements of SW480 tumor at 18 days post administration of tumor cells. FIG. 7C shows the images of solid tumors that were formed in balb/c nude mice. The growth of solid tumors in I_0.25%/G_3% was similar to Geltrex (P>0.05). The IVK8 peptide I_0.33% hydrogel did not result in tumor formation.

FIGS. 8A and 8B respectively show the volume and weight measurements of SW480 tumor at 14 days post administration of tumor cells. FIG. 8C shows the images of solid tumors that were formed in balb/c nude mice. The growth of solid tumors in I_0.25%/G_3% and I_0.25%/G_6% were similar to Geltrex (P>0.05). FIG. 8D shows the body weight of the mice on Day 0 (<1 min after injection) and Day 14 post administration of the cell-loaded hydrogels. FIG. 8E shows the H&E staining of the skin sections surrounding the SW480 tumors. Scale bar represents 200 μm.

FIG. 9 shows the live/dead staining of HepG2 spheroids cultured for 7 days in various hydrogel samples. The zone of viable cells appear as green, while the zone of nonviable cells appear as red. Scale bar represents 200 μm.

FIGS. 10A to 10E show the hydrogels of different compositions while FIG. 10F shows the tube without a sample.

FIGS. 11A to 11E show light microscopy images illustrating the transparency and clarity of the various hydrogels.

FIGS. 12A and 12B show the growth of SW480 tumor spheroids using other peptide/gelatin hydrogels. In FIG. 12A, for each day the size of the tumor spheroids is shown in the bar chart from left to right in the following order Geltrex, IVK8_0.25%/G_3%, IIK8_0.25%/G_3%, ILK8_0.25%/G_3%, IIK12_0.25%/G_3%, and IVK12_0.25%/G_3%.

FIGS. 13A to 13F show light microscopy images of SW480 tumor spheroids with different formulations of peptide/gelatin hydrogels at Day 7.

FIGS. 14A and 14B show images of IVK8_0.25%/PEI(25k)_3% which appears transparent and clear indicating that IVK8 is able to form suitable gels with other charged macromolecule like PEI (25k).

Some of the figures contain one or more panels, and the reference of the figure number and alphabet refers to the panel in the figure.

### DETAILED DESCRIPTION

In the following description, numerous specific details are set forth in order to provide a thorough understanding of various illustrative embodiments of the invention. It will be understood, however, to one skilled in the art, that embodiments of the invention may be practiced without some or all of these specific details. Embodiments described in the context of one of the methods or devices are analogously valid for the other methods or devices. Similarly, embodiments described in the context of a method are analogously valid for a device, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The term “agent” and “drug” are used herein, for purposes of the specification and claims, to mean chemical compounds, mixtures of chemical compounds, biological macromolecules, or extracts made from biological materials such as bacteria, plants, fungi, or animal particularly mammalian) cells or tissues that are suspected of having therapeutic properties. The agent or drug may be purified, substantially purified or partially purified.

The term “physiologically acceptable” defines a carrier or diluent that does not abrogate the biological activity and properties of the compound. The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, PA, 18th edition, 1990. The term “physiological conditions” refers to conditions typically found in organisms and cells, and typically refer to conditions with a temperature range of 20-40° C., atmospheric pressure of 1, pH of 6-8, glucose concentration of 1-20 mM, and atmospheric oxygen concentration

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides of the invention, although chemical or post-expression modifications of these polypeptides may be included or excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini, It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. [See, for instance Creighton. (1993), Posttranslational Covalent Modification of Proteins, W. H. Freeman and Company, New York B. C. Johnson, Ed., Academic Press, New York 1-12; Seifter, et al., (1990) Meth Enzymol 182:626-646; Rattan et al., (1992) Ann NY AcadSci 663:48-62]. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring. Various amino acids are described herein by its full name, and conventional 1-letter and 3-letter abbreviations as is known in the art.

Gelatin (or gelatine) is a mixture of peptides obtained from the hydrolysis of collagen, typically from the skin bones and connective tissues of animals such as cattle, chicken, pig and fish. The gelatin used herein may be from any suitable source.

The development of a peptide/gelatin hybrid hydrogel as an alternative to basement-membrane matrices such as Matrigel™ (BD, U.S.A.) and Geltrex™ (Thermofisher, U.S.A.) is described. Despite being commonly used for a diverse range of cell-culture applications, there are questionable issues relating to their usage, including the presence of xenogenic contaminants, complexity of their compositions as well as batch inconsistencies. These problems can be easily overcome with the peptide/gelatin hybrid hydrogel described herein due to the simplicity of its composition that can be precisely controlled. The absence of animal components from this material provides an added advantage over basement-membrane matrices with minimal interference to experimental outcomes. In addition, there are only two components in this hydrogel: 1) a short 8 or 12 amino acid self-assembling peptide and 2) gelatin, and this will enable the material to be manufactured with low production cost. When the peptide/gelatin hybrid hydrogels were used as scaffolds for in vitro 3D culture of tumor spheroids, they provided similar growth and viability of the cancer cells. Hypoxic, proliferative and apoptotic characteristics of the tumor spheroids were also similar between the peptide/gelatin hybrid hydrogels and Geltrex. Furthermore, in vivo expansion of solid tumors with peptide/gelatin hybrid hydrogels as supporting matrix was also evaluated, and the growth of tumors was similar between peptide/gelatin hybrid hydrogels and Geltrex in the mouse model. Importantly, the peptide/gelatin hybrid hydrogels showed excellent in vivo biocompatibility with no observable difference with regards to body weight and the wellbeing of the animals compared to those treated with Geltrex.

The viability of developing a hydrogel based material that is capable of providing an environment that can support and promote the expansion of cells for both in vitro 3D tumor spheroids cultures and in vivo tumorgrafts and the development of a scaffold prepared using gelatin and a self-assembling peptide IEVEIRVK (IVK8, SEQ ID NO: 2) is described. The key features of these peptides include regular alternating of hydrophobic and hydrophilic amino acids that encompass both arginine (R) and lysine (K), where the charges are arranged in the pattern (−−++). This arrangement enables the peptide to self-assemble into matrices with tunable mechanical strength and sustainable stability for a range of biomedical applications. Arginine and lysine are positively charged at physiological conditions or a pH of 7 to 7.4 at 25° C., while glutamic acid and aspartic acid are negatively charged. Isoleucine (I), valine (V) and leucine (L) were selected as hydrophobic residues as they have strong β-sheet folding propensity. To minimize charge repulsion, the peptides were amidated at the C terminus. As the N-terminus has a hydrophobic residue, it is not necessary to acetylate the N-terminus, but may be done by known methods. If the N-terminus has a hydrophilic residue, it may be functionalized by known methods for example acetylation to minimize charge repulsion.

In IVK8, R was selected for its high propensity for gelation while K was used in combination with R for its relatively lower cytotoxicity. In water, IVK8 exists in a random coil configuration and form peptide hydrogels (water retention ˜99%) at high concentrations. In the presence of salt or physiological conditions, the peptide self-assembles into β sheet conformation and form hydrogels of higher mechanical strength. Notably, rapid gelation is observed for IVK8 and the rate at which the gels form is temperature dependent. For instance, at a concentration of 2% wt/vol., IVK8 forms hydrogel within 60 minutes (min) at room temperature (23-24° C.), and the gelation can be accelerated to less than 30 min at 37° C.

Due to the absence of cell adhesion moieties in IVK8, it is unlikely for the peptide hydrogel to suffice as a 3D scaffold for cell proliferation. Thus, gelatin, a protein derived from collagen by controlled hydrolysis, was incorporated alongside with IVK8 to form a hybrid hydrogel to promote cell adhesion and growth. Gelatin can form hydrogels by chemical crosslinking. Given the multiple components present in gelatin, the reproducibility of the chemical modification may be challenging. The use of any added reagents and crosslinkers was circumvented with the development of a 3D scaffold with the interpenetrating network of self-assembling peptide IVK8 and gelatin. The hydrogel was formed by simple physical mixing of the two components, IVK8 and gelatin, and leaving it to stand at 37° C. for 15 min. This procedure is both time and cost-saving, and the composition of the hydrogel is well-defined and consistent. The hydrogel was studied in both in vitro and in vivo for its capacity in supporting cell growth and establishment of tumor models. 3D cell culture was performed by encapsulating tumor spheroids in the hydrogels, and cell viability assays and size measurements showed that the cells in the tumor spheroids were able to proliferate over time. Importantly, the growth of tumor spheroids and in vivo tumors in the IVK8/gelatin hydrogels were equivalent to the commercially available basement membrane product. Geltrex (Thermofisher, U.S.A.). The penetration of a small molecule model drug, doxorubicin, was also similar between the IVK8/gelatin hybrid hydrogels and Geltrex. Given the low cost of production for the IVK8/gelatin hydrogels and definitive composition, this hydrogel stands out as a promising alternative to commercial cell culture matrices such as Matrigel and Geltrex. Other self-assembling peptides may also be used in place of IVK8, for example IEIEIRIK (IIK8, SEQ ID NO: 3), IELEIRLK (ILK8, SEQ ID NO: 4), IRVKIEVEIRVK (IVK12. SEQ ID NO: 5), IRIKIEIEIRIK (IIK12, SEQ ID NO: 6), IRVEIRVEIRVE (IRV12, SEQ ID NO: 7), IEVEIEVKIRVK (IEV12, SEQ ID NO: 8) and peptides with the reverse sequence. The sequences of the peptides that may be used are shown in Table 2. The formation of a hydrogel by these peptides have been previously reported in WO2021015675 which is incorporated by reference in its entirety herein.

### METHODS

### Materials

The peptides IVK8, IIK8, ILK8, IVK12, and IIK12 were synthesized by GL Biochem (Shanghai, China) with ≥95% purity determined using analytical reverse phase high-performance liquid chromatography (RP-HPLC). Gelatin was purchased from Sigma Aldrich (U.S.A.) and CellTiter 96® Aqueous One Solution Cell Proliferation Assay (MTS) was purchased from Promega and used as according to the manufacturers' instructions. Human hepatocellular carcinoma HepG2 and human colorectal adenocarcinoma SW480 cell lines were purchased from ATCC, U.S.A. and anti-cancer drug, Doxorubicin (DOX) was purchased from MedChemExpress, U.S.A.

### Preparation of Hydrogels

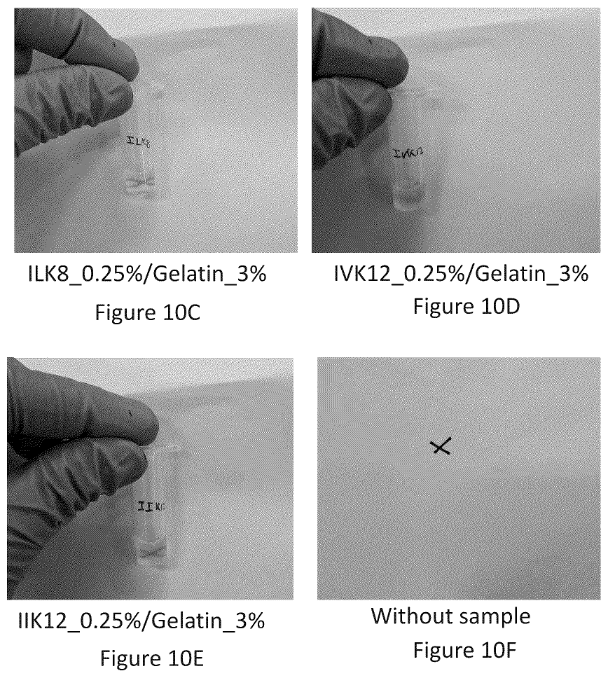

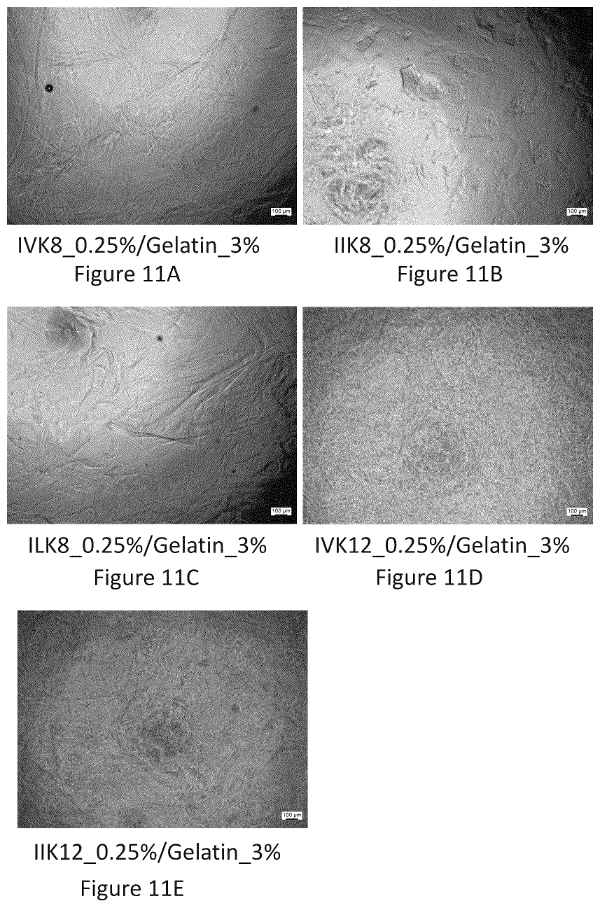

IVK8 was dissolved in 300 mM sucrose sterile solution at a peptide concentration of 1.0% wt/vol. and stored at 4° C. until required. The peptide remains a solution at cold conditions (4° C. or ice bath). Gelatin was dissolved using sterile DI water at 60 mg/ml at 60° C. To form the hydrogel, gelatin was removed from the warm water bath and the cold IVK8 solution was added to the gelatin. DMEM was then added to this mixture at a volume ratio of 3:7 DMEM: mixture and was left to stand at 37° C. for 10 min for gelation to occur. The procedure for preparing IVK8 hydrogels was the same. Once formed, the gels remain stable at temperatures between 25 to 37° C. Similarly, the other peptide/gelatin hydrogels may be prepared by the same method described for IVK8, further detail and modifications of the general procedure may be found in WO2021015675. For example. IIK8, ILK8, IVK12, and IIK12 may be used instead of IVK8 to prepare the peptide/gelatin hydrogel. It may be observed from FIGS. 10A to 10 that shorter peptides result in clearer hydrogels. IVK8_0.25%/Gelatin_3% (FIG. 10A) and IIK8_0.25%/Gelatin_3% (FIG. 10B) are the clearest and most transparent, followed by ILK8_0 25%/Gelatin_3% (FIG. 10C). IVK12 0.25%/Gelatin_3% (FIG. 10D) is the most opaque and unclear of the five hydrogels shown. IVK8_0.25%/Gelatin_3% (FIG. 11A) and ILK8_0.25%/Gelatin_3% (FIG. 11C) shows the greatest transparency and clarity of the hydrogels. Some micro-clustering/aggregation was seen in IIK8_0.25%/Gelatin_3% (FIG. 11B). The high opacity of IVK12_0.25%/Gelatin_3% (FIG. 11D) and IIK12_0.25%/Gelatin_3% (FIG. 11E) may make them less suitable for 3D culture of tumor spheroid due to difficulties in observing the spheroids.

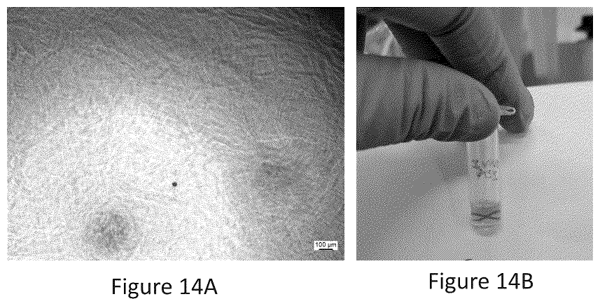

FIGS. 14A and 14B show images of IVK8_0.25%/PEI(25k)_3% which appears transparent and clear indicating that IVK8 is able to form suitable gels with other charged macromolecule like polyethylenimine (PEI, molecular weight 25k). PEI is partially charged at a pH of 7.4. The G′ (storage modulus) and G″ (loss modulus) values of the IVK8_0.25%/PEI(25k)_3% hydrogel are shown in FIG. 14C.

The concentration of the solutes (e.g. peptides and salts) herein may be provided in weight by volume percent (and may be abbreviated as (% w/v), (w/v %), or (%) herein, and is equivalent to the mass of solute in grams dissolved in 100 mL of solvent. Alternatively, wt % (or wt. %) may be used to mean the same as the solvent is water and assumed to have a density of 1 g/cm³.

### Rheological Studies of Hydrogels

Vial tilting method was applied to determine the gelation time of the hydrogels. When the sample showed no flow within 5 seconds(s), it would be considered as a completely formed hydrogel. Rheology experiments were performed on a control-strain rheometer (ARES G2, TA instruments), as described previously. Briefly, a parallel plate measuring geometry (8 mm diameter, gap 1 mm) with oscillatory strain of 1% and the dynamic storage modulus (G′) and loss modulus (G″) were examined as a function of frequency from 1 to 100 rad/s. In addition, a flow sweep to study the viscosity changes as a function of shear rate of hydrogel samples.

### Enzymatic Degradation of Hydrogels

Hydrogel samples (400 μL) were first prepared and transferred to 4 mL glass vials. After which. 2 mL of PBS containing collagenase Type IV (Gibco, U.S.A.) (0.01 wt. % incubated at 37° C. was added. At regular intervals, the enzyme-containing PBS media were removed from the hydrogel, and the weight of degraded hydrogel (W_{d}) was determined. After weighing, fresh enzyme-containing PBS media were added to the samples. The relative amount of hydrogel remaining in the vials was calculated using the following equation:

Relative amount of hydrogel left=W_{d}/Wᵢ where W_{d} and Wᵢ are the weights of the degraded and original hydrogels, respectively.

### In Vitro Culture of Tumor Spheroids in Hydrogels

Human hepatocellular carcinoma HepG2 and human colorectal adenocarcinoma SW480 were cultivated in Dulbecco's Modified Eagle Medium (DMEM), supplemented by 10% Fetal Bovine Serum and 1% Penicillin, incubated at 37° C., 5% CO₂. To prepare the tumor spheroids, 200 μL of cell suspension (3500 cells/mL) were seeded per well onto 96-well ultralow adhesion round bottom plates (Corning, U.S.A.). After which, the plate was centrifuged for 5 min at 1500 rpm) and incubated at 37° C. for 2-3 days for the cells to assemble into tumor spheroids. The IVK8/gelatin hydrogels were then prepared according to the steps in Section 2.2 and was used right after mixing the peptide and gelatin together. A positive control group was set up using the basement membrane product, Geltrex™ (Thermofisher, U.S.A.). Geltrex was kept in an ice bath during the experiment. The hydrogels were transferred to the tumor spheroids by first removing 150 μL of the spent medium. Subsequently, 70 μL of the hydrogels were added per well and the samples and incubated at 37° C. for 30 min before 100 μL were added to each well. The spent medium was replaced once every 2 to 3 days by removing 80 μL medium and adding 80 μL fresh medium. The growth of the tumor spheroids was monitored over time using DMil inverted light microscope (Leica, U.S.A.).

### Cell Viability Assay

The viability of tumor spheroids cultured in the hydrogels for 7 days was determined by the CellTiter 96 AQueous One Solution Cell Proliferation Assay (MTS) (Promega, U.S.A.). Briefly, 80 μL of the spent medium was removed from each well containing the tumor spheroids. Next, 80 μL containing DMEM: MTS at 1:1 volume ratio was then added back into the wells. The plate was then incubated at 37° C. for 4.5 h. After which, the medium was pipetted several times to break up any aggregates that may be present and 100 μL of this solution was transferred to a fresh 96 well plate. The absorbance of the samples was measured at wavelength 490 nm using a microplate reader (Tecan, Switzerland).

### Distribution of Live and Dead Cells in Tumor Spheroids

The distribution of live and dead cells in spheroids cultured in hydrogels for 7 days was analyzed. On the day of imaging, 100 μL of the spent medium was removed and 125 μL of live/dead stains from the Live/dead Cell Imaging Kit (488/570) (Thermofisher, U.S.A.) was added to the samples. Next, the samples were incubated for 4 hours at 37° C., washed 5 times with PBS by removing 80 μl medium and adding 80 μl of fresh PBS. The spheroids were then imaged using the IX81 HCS microscope (Olympus, Japan).

### Hypoxia Detection in Tumor Spheroids

Hypoxia was detected in spheroids cultured in hydrogels for 7 days using the Image-It Hypoxia Red Reagent (Thermofisher, U.S.A.). Briefly, DMEM containing Image-It Hypoxia Reagent (10 μM) and NucBlue Live Cell Stain (4 drops per mL of DMEM) (Thermofisher, U.S.A.) was first prepared. After which, 80 μL of the spent medium was removed from the samples and the prepared staining solution was added. Next, the samples were incubated at 37° C. for 3 hours, washed 4 times by removing 80 μl medium and adding 80 μl of fresh PBS repeatedly. The spheroids were then imaged using the IX81 HCS microscope (Olympus, Japan).

### Distribution of Proliferative and Apoptotic Cells in Tumor Spheroids Via Immunohistochemistry Analysis

The distribution of proliferative and apoptotic cells in spheroids cultured in hydrogels for 7 days was analyzed. First, the samples were washed 3 times by removing 80 μL medium and adding 80 μL of fresh PBS repeatedly. At the last wash, fresh PBS was not added but 50 μL more PBS was removed. Next, 10% neutral buffered formalin (NBF) (200 μL) was added to the sampled and subsequently incubated at room temperature for 4 hours. During which, agarose was dissolved in distilled water at 95° C. and kept warm at 65° C. Thereafter, agarose embedding was performed by transferring the spheroids from each well to embedding molds. 250 μL of agarose solution was then added into the mold and incubated at 4° C. for 30 min. The agarose block was then transferred to tissue processing cassettes and kept in 10% NBF followed by paraffin embedding procedures. Proliferative and apoptosis status of the cells were detected using anti-Ki-67 rabbit polyclonal (Abcam. U.S.A.) and anti-Cleaved rabbit Caspase-3 polyclonal antibodies (Abcam. U.S.A.). For histological examination, the samples were fixed in 4% neutral buffered formalin and then stained with hematoxylin-eosin (H&E) using standard techniques. The slides were imaged using Axio Imager microscope (Carl Zeiss, Germany).

### Treatment of Tumor Spheroids Using Model Anti-Cancer Drug, Doxorubicin

To study the effects of commonly used anti-cancer therapeutics in the 3D tumor spheroids, a model drug commonly used for cancer treatment, Doxorubicin (DOX), was used.

Spheriod size changes and Cell Viability: Doxorubicin was first dissolved in sterile distilled water to form a stock solution at 1 mg/mL. This solution was then diluted using DMEM medium to DOX concentrations of 0.8, 4, 20, 100 uM. Next, 80 μL of spent medium was removed from tumor spheroids that had been cultured in hydrogels for 7 days and 80 μL of DMEM containing DOX was added. Then the samples were incubated at 37° C. for 24 hours. The size difference in tumor spheroids were monitored using DMil inverted light microscope (Leica, U.S.A.) and cell viability assay was performed as in Section 2.5.

### In Vivo Tumor Growth Using IVK8/Gelatin Hybrid Hydrogels as Supporting Matrix

All animal experiments were conducted in accordance with the approved protocol from the Institutional Animal Care and Use Committee (IACUC) at the Biological Resource Centre of Singapore. Female balb/c nude mice, weighing 18-22 g were injected with 200 μL of a cell suspension with either IVK8/Gelatin hydrogels (3:7 volume ratio of cells in DMEM: IVK8/Gelatin hydrogel) or Geltrex (1:1 volume ratio of cells in DMEM: Geltrex) (Thermofisher, U.S.A.) containing 1.0×10⁷ SW480 cells subcutaneously using 23G syringe needles. Two studies were conducted. In the first study, three groups of gel formulations: 1) Geltrex, 2) I_0.25%/G_3% and 3) I_0.33% were tested on 4 mice per group. In the second study, 1) Geltrex, 2) I_0.25%/G_3% and 3) 1_0.25%/G_6% were tested on 5 mice per group. The tumor size was measured using calipers in two orthogonal diameters and the volume was calculated as L×W²/2, where L and W are the major and minor diameters respectively. The data was analyzed using two-tailed Student's T-test to statistically evaluate the difference in tumor volumes. Statistical significant values were defined as P≤0.05. In addition, the mice from the 2^{nd} study were sacrificed and the skin tissue at the area surrounding the tumors were excised and histological sectioning was performed. The samples were stained using H&E and imaged using Axio Imager microscope (Carl Zeiss, Germany).

### Statistical Analysis

All measurements were expressed as average±standard deviation. Comparison between different groups was evaluated using the two-tailed Student's T-test. Statistical significant values were defined as P≤0.05.

### RESULTS AND DISCUSSION

### Peptide Design and Characterization

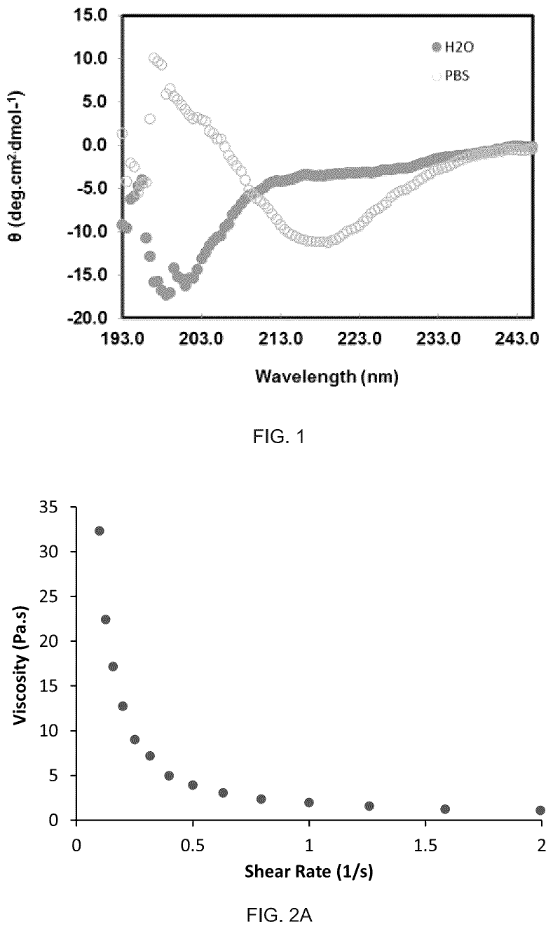

The development of a scaffold prepared using gelatin and a novel self-assembling peptide IEVEIRVK (IVK8, SEQ ID NO: 2) is described herein. IVK8 consists of 8 natural amino acid residues with periodic repeats of charged hydrophilic (isoleucine (I) and valine (V)) and hydrophobic (arginine (R) and lysine (K)) amino acids. Isoleucine (I) and valine (V) were chosen as hydrophobic residues because of their strong β-sheet folding propensity, while arginine (R) was selected for its faster and stronger gelation behavior and K was used in combination with R for its relatively lower cytotoxicity. As shown in FIG. 1, the CD spectrum of IVK8 in water displayed a minimum at 198 nm, indicating that it is of a random coil confirmation. This is likely attributed to the intermolecular electrostatic repulsion between the protonated R and K residues. However, in the presence of a salt such as PBS, IVK8 folded and self-assembled into a β-sheet conformation as characterized by the maximum absorption at ˜197 nm and a strong negative ellipticity at ˜219 nm. The presence of salt in the buffer triggers the self-assembly of the peptide by shielding and neutralizing the charges, and enables the hydrophilic residues E, R and K to lie on the same side with complementary ionic interactions derived from positively and negatively charged ionic groups.

### Rheological Properties of IVK8/Gelatin Hybrid Hydrogels

The mechanical strength and gelation time at 37° C. of the IVK8/gelatin hybrid hydrogels with different concentrations of peptide and gelatin were evaluated (Table 1). For hydrogels that were prepared with IVK8 solely, the gel strength corresponds to the peptide concentration of 1_0.25% (G′=210 Pa), I_0.33% (G′=382 Pa) and I_1.0% (G′=423 Pa). Similarly, for IVK8/gelatin hybrid hydrogels, the increase in peptide concentration at the same gelatin concentration results in higher gel strength, for example: I_0.33%/G_2% (G′ =226 Pa, gelation time 10 min) versus I_0.67%/G_2% (G′=562 Pa, gelation time 5 min). This is possibly due to more peptide molecules participating in physical cross-linking at higher concentrations. On the contrary, Geltrex showed the lowest mechanical strength with G′=40 Pa. ILK8_0.25%/G_3% had similar gel strength to the corresponding IVK8 hydrogel. The hydrogels prepared using the other peptides had lower gel strength, which may be increased by increasing the concentration of the peptide used to form the hydrogel. Table 2 shows the amino acid sequences of the peptides used herein.

TABLE 1
G′ (storage modulus) and G″ (loss modulus) values of peptide-gelatin
hybrid hydrogels self -assembled from IVK8 and gelatin at frequency of 1 Hz, 1% strain.

| | Peptide | Peptide | Gelatin | Gelation | G′ | G″ | tan (δ) = |
|---|---|---|---|---|---|---|---|
| Sample | Name | (wt. %) | (wt. %) | Time (min) | (Pa) | (Pa) | G″/G |
| I_0.67%/G_2% | IVK8 | 0.67 | 2 | 5 | 562 | 138 | 0.246 |
| I_0.5%/G_3% | IVK8 | 0.5 | 3 | 5 | 540 | 150 | 0.278 |
| I_0.33%/G_2% | IVK8 | 0.33 | 2 | 10 | 226 | 106 | 0.469 |
| I_0.25%/G_3% | IVK8 | 0.25 | 3 | 10 | 200 | 73 | 0.365 |
| I_0.25% | IVK8 | 0.25 | 0 | 10 | 210 | 100 | 0.476 |
| I_0.33% | IVK8 | 0.33 | 0 | 10 | 382 | 119 | 0.312 |
| I_1% | IVK8 | 1 | 0 | 5 | 423 | 71 | 0.168 |
| IIK8_0.25%/G_3% | IIK8 | 0.25 | 3 | 10 | 56 | 26 | 0.464 |
| ILK8_0.25%/G_3% | ILK8 | 0.25 | 3 | 10 | 206 | 51 | 0.248 |
| IIK12_0.25%/G_3% | IIK12 | 0.25 | 3 | 30 | 23 | 5 | 0.217 |
| IVK12_0.25%/G_3% | IVK12 | 0.25 | 3 | 30 | 38 | 5 | 0.132 |
| IVK8_0.25%/PEI_3% | IVK8 | 0.25 | 3 (PEI | 5 | 243 | 52 | 0.206 |
| | | | instead | | | | |
| | | | of | | | | |
| | | | gelatin) | | | | |
| Geltrex | | | | 10 | 40 | 21 | 0.525 |

*Higher tan(δ) values indicates hydrogel with lower mechanical strength.

TABLE 2
Peptide Sequence

| | SEQ ID | Peptide |
|---|---|---|
| Name | NO: | Sequence |
| IVK8 | 2 | IEVEIRVK |
| IK8 | 3 | IEIEIRIK |
| ILK8 | 4 | IELEIRLK |
| IVK12 | 5 | IRVKIEVEIRVK |
| IK12 | 6 | IRIKIEIEIRIK |
| IRV12 | 7 | IRVEIRVEIRVE |
| IEV 12 | 8 | IEVEIEVKIRVK |
| Reverse IVK8 | 9 | KVRIEVEI |
| Reverse IIK8 | 10 | KIRIEIEI |
| Reverse ILK8 | 11 | KLRIELEI |
| Reverse IVK12 | 12 | KVRIEVEIKVRI |
| Reverse IIK12 | 13 | KIRIEIEIKIRI |
| Reverse IRV12 | 14 | EVRIEVRIEVRI |
| Reverse IEV12 | 15 | KVRIKVEIEVEI |

The peptides of SEQ ID No. 2 to 8 were amidated at the C terminus. The peptides of SEQ ID No. 9 to 15 were amidated at the C-terminus and N-acetylated at the N-terminus.

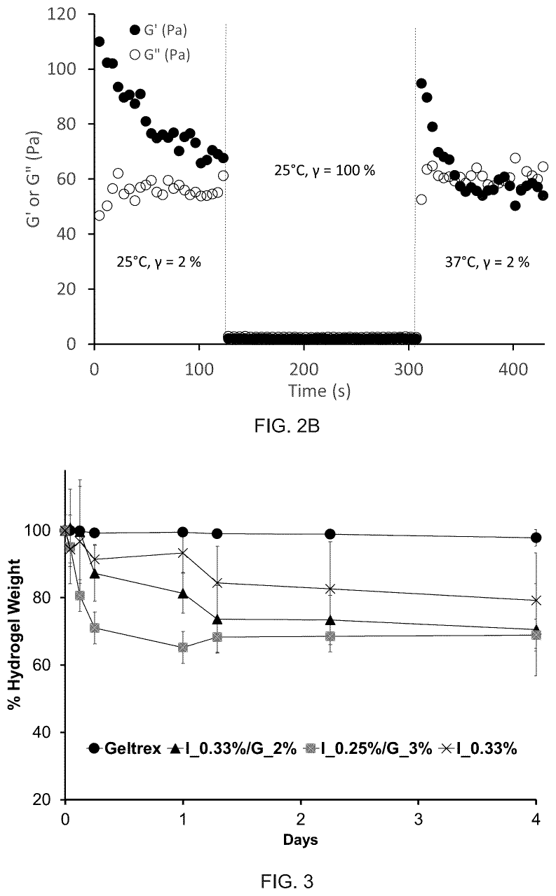

Shear-thinning property is crucial for the IVK8/gelatin hybrid hydrogels to allow it to function as an injectable matrix to support cell growth in vivo. FIG. 2A shows that viscosity of I_0.25%/G_3% reduces drastically with increasing shear rate, thereby demonstrating the shear-thinning behavior of the hybrid hydrogel. The drop in viscosity with greater shear rate is due to the disruption of physical cross-links between the peptide and gelatin molecules upon the application of shear stress. After delivery to the site of injection, it is crucial for the disrupted hydrogel to recover its mechanical properties. The recovery of disrupted hydrogels was simulated using a dynamic step strain amplitude test. The hybrid hydrogel was mixed with DMEM at a volume ratio of 7:3 to simulate the incorporation of cell suspension. As illustrated in FIG. 2B, the hydrogel of I_0.25%/G_3% has an initial G′ of ˜80 Pa when low strain of 2% at 25° C. was applied. However, when subjected to a high strain (100%), the G′ value rapidly decreased by 40-folds to ˜2 Pa and G′/G″ increased to >1, indicating the predominance of liquid-like behavior of the hydrogel. After being subjected to continuous high strain for 3 min, the strain was reduced to 2% with the temperature being increased to 37° C. to simulate in vivo conditions right after injection. Notably, the storage modulus G′ was recovered immediately, thereby showing the reversibility in rheological behavior. The rapid recovery kinetics may be attributed to the quick formation of physical cross-links (e.g. hydrogen bonding, electrostatic interactions and hydrophobic interactions) within the peptide and gelatin network right after the removal of the high strain conditions.

### Enzymatic Degradation of IVK8/Gelatin Hybrid Hydrogel

The Enzymatic Degradation Of Hydrogels In The Presence of Collagenase Type IV (gelatinase) was investigated. This enzyme is able to cleave peptide bonds in gelatin, and is found ubiquitously in the body as part the control of the immune defense system. It was observed that the IVK8/gelatin hybrid hydrogels degraded much faster compared to Geltrex. As seen in FIG. 3, the I_0.25%/G_3% hydrogel showed faster degradation compared to the I_0.33%_G2% hydrogel. thereby showing that higher gelatin content led to quicker proteolytic degradation of the hybrid hydrogels. Within the first 24h, the I_0.25%/G_3% hydrogel lost 35% of its weight while the I_0.33%_G2% hydrogel lost 20% of its weight. The degradation of IVK8 hydrogel I_0.33% is lesser than the IVK8/gelatin hybrid hydrogels where only 7% of its weight was lost for the first 24 h. There is little degradation of Geltrex at ˜3% throughout the 4 days of incubation.

### In Vitro Culture of Tumor Spheroids in IVK8/Gelatin Hybrid Hydrogels

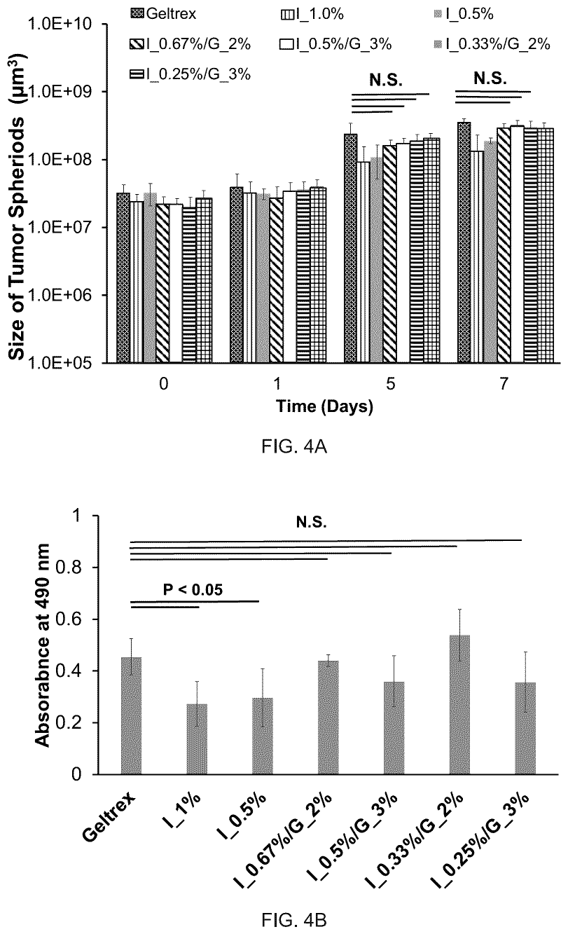

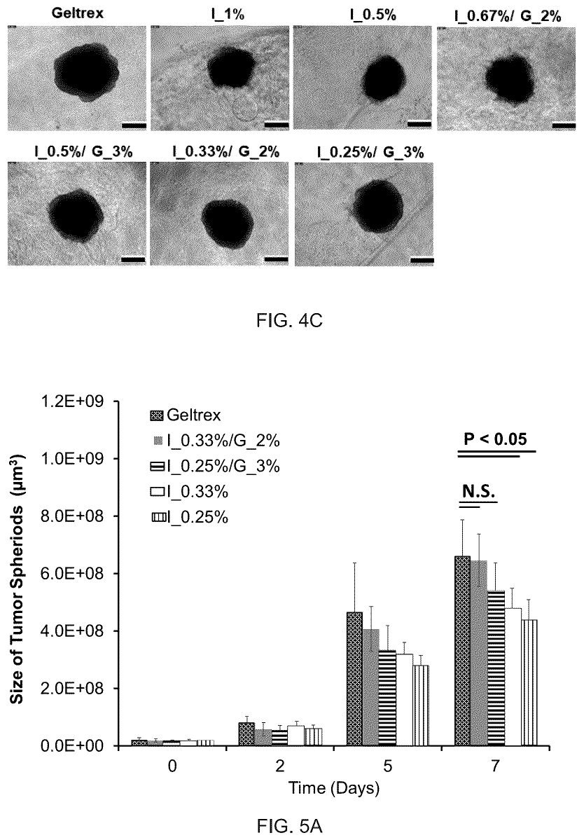

The potential of using IVK8/gelatin hybrid hydrogels as a 3D scaffold was examined using HepG2 and SW480 tumor spheroids cultures. From FIG. 4, it may be seen the HepG2 tumor spheroids that were cultured for 7 days in the I_0.5%/G_3% hydrogel, the I_0.25%/G_3% hydrogel and the I_0.33%/G_2% hydrogel were of similar size compared to those cultured in Geltrex (P>0.05). However, spheroids that were cultured in hybrid hydrogel with higher IVK8 and Gelatin concentration (I_0.67%/G_2% hydrogel) were significantly smaller compared to those cultured in Geltrex. Notably, IVK8/gelatin hybrid hydrogels were highly suitable for culturing the tumor spheriods as there was no significant difference in cell viability for tumor spheroids cultured in all four hybrid hydrogel formulations as compared to Geltrex (P>0.05). Peptide hydrogels prepared using IVK8 solely were unsuitable in supporting the growth of the tumor spheroids as the size of the spheroids were significantly smaller and the cells were significantly less viable compared to those cultured in Geltrex.

From this assessment, two IVK8/gelatin hybrid hydrogels were identified that were optimal for the culture of tumor spheroids with regards to cell viability, growth of the spheriods, and at the same time, taking into consideration of the amount of material required: 1) I_0.25%/G_3% and 2) 1_0.33%/G_2%.

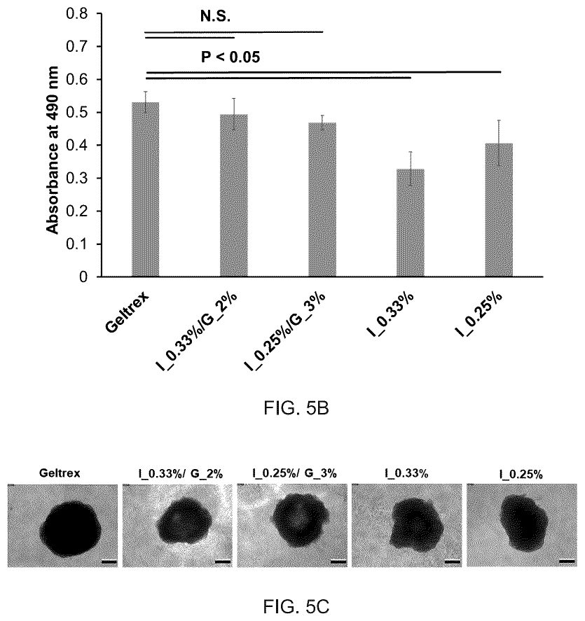

To evaluate whether the hybrid hydrogels on tumor spheroids could be extended to other cancer cell types: colorectal cancer cells SW480, was cultured using the two aforementioned IVK8/Gelatin hydrogels. Importantly, SW480 tumor spheroids that were cultured for 7 days in both hybrid hydrogels I_0.25%/G_3% and I_0.33%/G_2% were of similar size compared to those cultured Geltrex (P>0.05) (FIGS. 5A-C). Similarly, the growth of the tumor spheroids cultured in IVK8 hydrogels I_0.33% and I_0.25% were significantly slower compared to those culture in Geltrex (P<0.05).

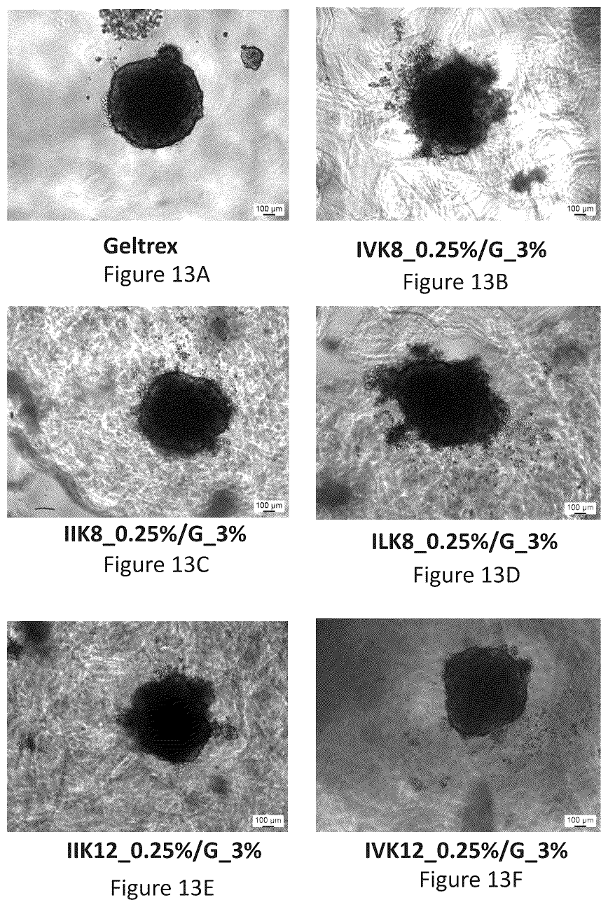

FIG. 13 shows the light microscopy images of the SW480 tumor spheroids on Day 7. It may be observed that the peptide/gelatin hybrid hydrogels formed using IVK8 (FIG. 13B) and IIK8 (FIG. 13C) are the least opaque. The peptide/gelatin hybrid hydrogels formed using ILK8 (FIG. 13D) and IIK12 (FIG. 13E) are more opaque than those formed using IVK8 and IIK8. IVK12 (FIG. 13F) is the most opaque amongst all the formulations, possibility due to precipitation of the gel constituents.

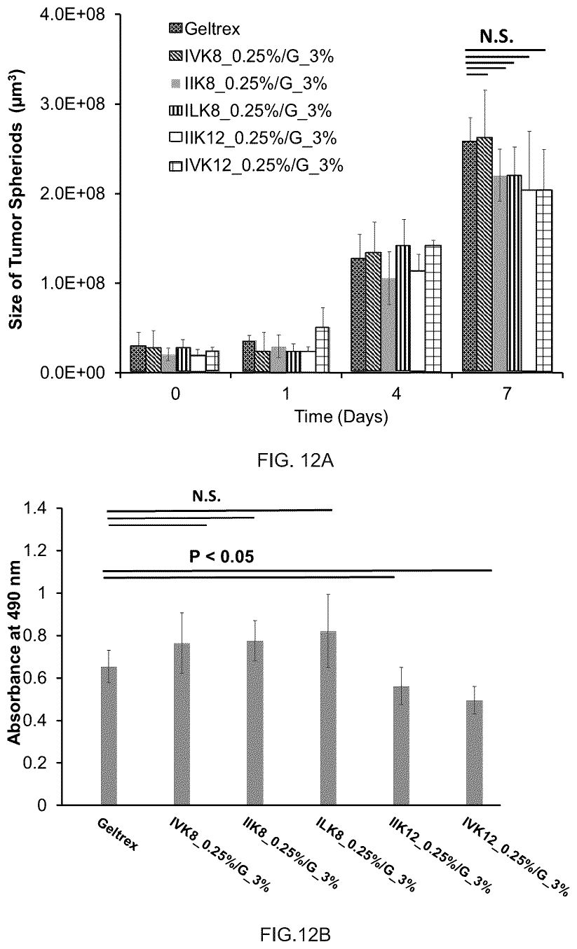

A comparison of using hydrogels made with the different peptide is shown in FIGS. 12A and 12B. From FIG. 12A, no significant difference in the size of the SW480 tumor spheroids were observed between the hydrogels of the various formulations and Geltrex.

### In Vitro 3D Tumor Models Cultured in IVK8/Gelatin Hybrid Hydrogels

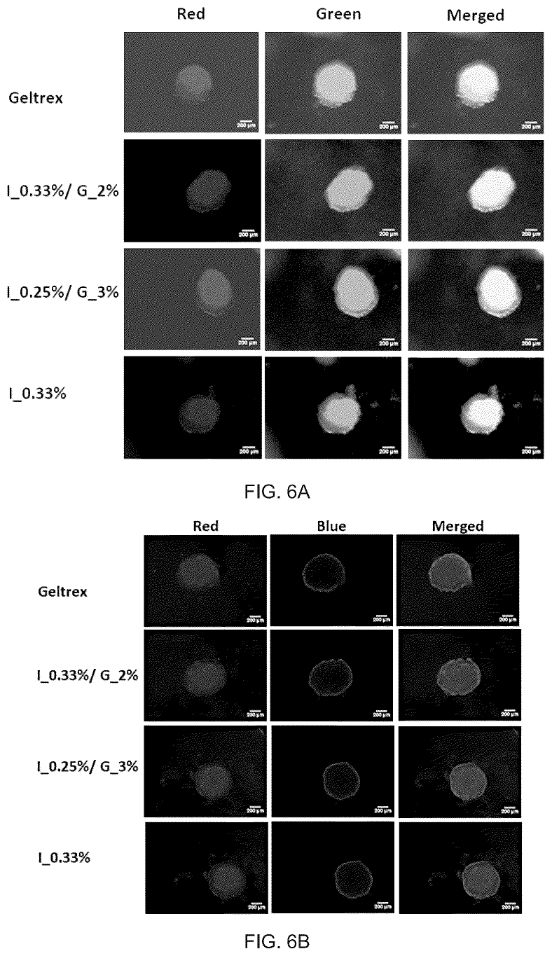

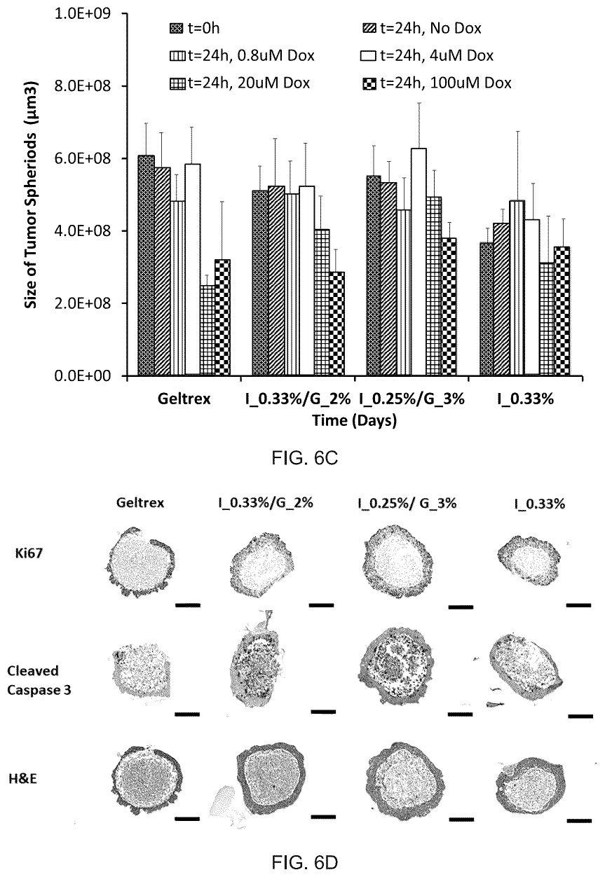

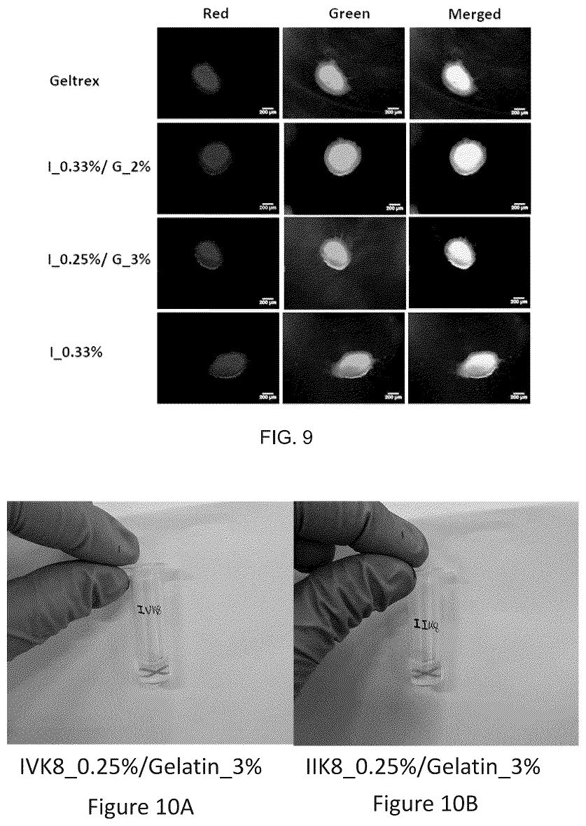

The variations in pH, nutrients, metabolites and oxygen contents in different radial zones of tumor spheroids results in observable differences in the viability of cells within the spheroids. For both HepG2 and SW480, the dead or dying cells are present mostly in the core of the spheroids while the living cells are present throughout the spheroids (FIGS. 6A and 9). At the periphery region, there are more viable cells than dead/dying cells as indicated by the relatively higher intensity of green fluorescence versus red fluorescence. This observation is similar for both the IVK8/Gelatin hydrogels and Geltrex. For IVK8 peptide hydrogel I_0.33%, the zone of dead/dying cells is present throughout the spheroids, not only in the core region. This could be due to higher mechanical strength of the peptide hydrogel that reduces the transport of nutrients, oxygen and metabolites across the hydrogel barrier, thereby lowering the viability of the tumor cells.

It has been reported that as the growth of tumor spheroids exceeds 400 μm in diameter, they will develop a hypoxic core and activate survival signaling pathways and induce the expression of hypoxia inducible factors (HIF) to maintain cell viability tumors. In vivo, HIF proteins transcriptionally promote angiogenesis to gain nutrients and oxygen and regulate pH of the microenvironment. Hypoxia of SW480 spheroids cultured for 7 days in various hydrogel samples was detected using the Image It Hypoxia Reagent. It is non-fluorescent under non-hypoxic conditions and emits red fluorescence under hypoxic conditions. From FIG. 6B, the core of the spheroids appears red as low oxygen levels is detected, while the periphery is non-hypoxic and does not emit red fluorescence. The cells are counterstained with NucBlue Live ReadyProbes Reagent (Hoechst 33342) which emits blue fluorescence. The hypoxia regions occur at the core of the spheroids and do not differ between Geltrex, IVK8/Gelatin hybrid hydrogels as well as IVK8 peptide hydrogel.

In order to validate the culture of tumor spheroids in IVK8/Gelatin hybrid hydrogels as an in vitro model for drug testing, the penetration of a typical small molecular drug, Doxorubicin (Dox) was studied. FIG. 6C shows that there was a decreasing trend for the size of tumor spheroids cultured in Geltrex and IVK8/Gelatin hybrid hydrogels. However, there was no observable reduction in the size of the tumor spheroids for those cultured in IVK8 peptide hydrogel I_0.33%. This could be either due to impedance to drug penetration caused by the networks in the peptide hydrogel or the difference in cellular state (i.e. more dead/dying cells as seen in FIG. 6A) of the tumor spheroid compared to the other hydrogels.

To identify the areas of different cellular activities within the tumor spheroids, immunohistochemical staining for Ki67 and Cleaved Caspase 3 which are indicators for proliferation and apoptosis respectively was conducted. Importantly, majority of the detection of Ki67 occurs at the periphery of the spheroids while most of the Cleaved Caspase 3 is found at the core of the spheroids. The proliferative and apoptotic regions appear similar between Geltrex, IVK8/Gelatin hybrid hydrogels and IVK8 peptide hydrogel (FIG. 6D). H&E staining of the tumor spheroids shows a huge difference with respect to cell density at different region; where it is higher at the periphery and much lower at the core. A similar trend was observed for all the various gel formulations.

### In Vivo Expansion of Tumors With IVK8/Gelatin Hybrid Hydrogels as Supporting Matrix

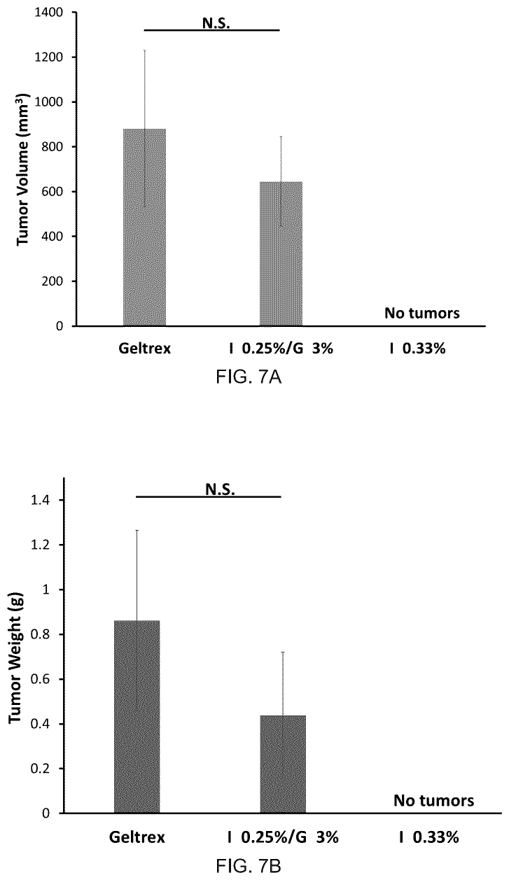

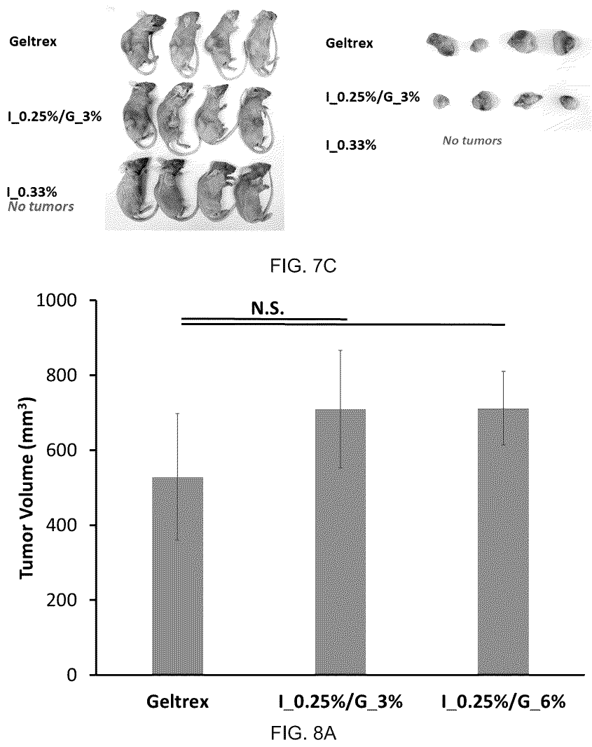

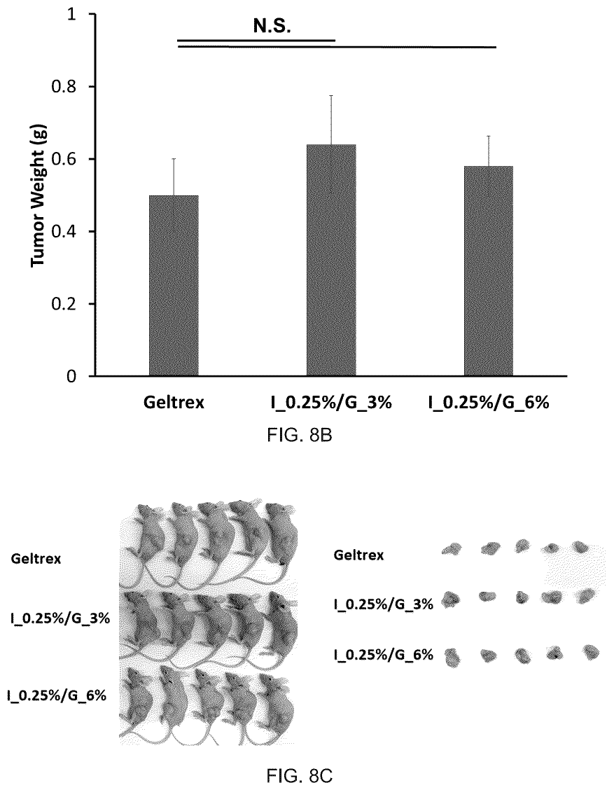

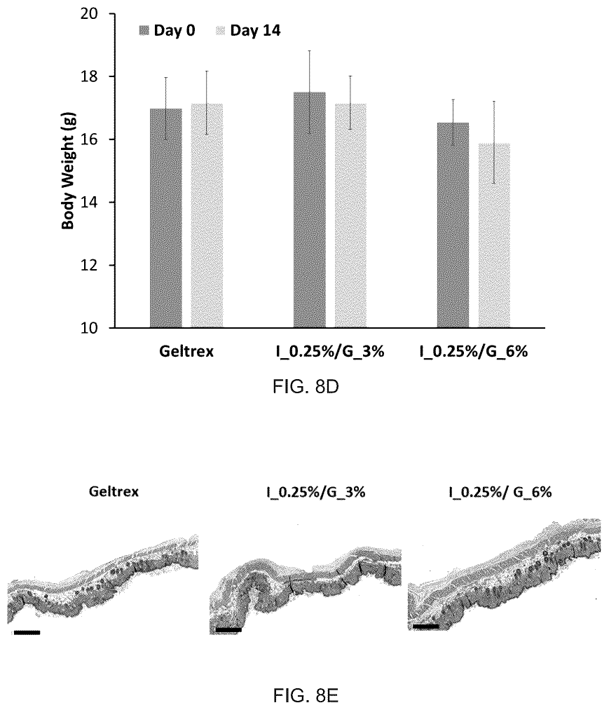

The growth of SW480 tumors using the IVK8/Gelatin hybrid hydrogel and IVK8 peptide hydrogel was tested and compared against Geltrex in mice model. FIGS. 7A-C showed that the tumor growth is similar between I_0.25%/G_3% hydrogels and Geltrex as both tumor volume and weight measurements showed no significant difference (P>0.05). With IVK8 peptide hydrogel I_0.33%, the tumor cells did not form solid tumors, and this demonstrates the importance of incorporating gelatin into the hybrid hydrogels for promoting cell growth in an in vivo environment. In the second study, the concentration of gelatin in the IVK8/gelatin hybrid hydrogels was increased from 3 wt. % to 6 wt. % but this did not further promote tumor growth (FIGS. 8A-C) There was also no significant difference between I_0.25%/G_3%, I_0.25%/G_6% hydrogels and Geltrex with respect to tumor growth. Notably, the hydrogels did not cause any body weight loss in the animals, suggesting that the IVK8/gelatin hybrid hydrogels is biocompatible for in vivo application (FIG. 8D). Furthermore, H&E staining of the skin sections surround the tumors showed no observable difference in the structure and morphology of the dermal tissue, thereby demonstrating that the IVK8/Gelatin hybrid hydrogels are biocompatible and non-inflammatory.

From FIG. 12A, no significant difference may be observed in the size of tumor spheroids between the various peptide-gelatin hydrogel formulations compared to Geltrex. From FIG. 12B, it may be observed that there was no significant difference in cell viability (at Day 7) of tumor spheroids culture using hybrid hydrogels formed using the shorter peptides IVK8. IIK8 and ILK8, but significantly lower viability may be observed for those cultured using longer peptides IIK12 and IVK12.

### Physical Properties of IVK8/Gelatin Hybrid Hydrogels

The mechanical properties of cell culture scaffolds can provide strong influence on the changes to cellular behavior. Such applications include directing stem cell differentiation and modelling cancer cell activity in relation to the stiffness of extracellular matrices From Table 1, it was demonstrated that the mechanical strength of the IVK8/gelatin hybrid hydrogels can be easily adjusted with changes to the peptide and gelatin concentrations. Hence, applying these materials on studying the dependency of cellular activity on material stiffness is a viable option.

The study of proteolytic degradation of IVK8/gelatin hybrid hydrogels showed that higher gelatin content led to quicker proteolytic degradation of the hybrid hydrogels (FIG. 3). Through intercellular interactions, tumor cells can cause changes in expression levels of adhesion factors and/or due to the elevated levels of secreted proteolytic enzymes, including collagenases. The enzymes break down extracellular matrices and enable tumors to change shape, detach and meander through interstitial spaces to cause disorder to the normal tissue architecture. Interestingly, collagenase did not cause substantial degradation of Geltrex, possibly due to the low concentration of collagenase used.

### Growth and Characterization of Tumor Spheroids in IVK8/Gelatin Hybrid Hydrogels

Tumor spheroids are clusters of cancer cells that represent the simplest in vitro model of solid tumors. These spheroids are prepared based on the inherent nature of epithelial cancer cells to form adhesive linkages between cells and thereby self-assemble into dense aggregates on a non-adherent surface or 3D scaffold. The properties of tumor spheroids closely resemble that of in vivo avascular tumors with regards to proximal cell-cell interactions, interactions with extracellular matrix (ECM), cellular response towards pH, nutrients, oxygen, and metabolite concentration gradients in a 3D clustered environment. Such 3D models provide the capability to mimic the behavior of in vivo tumors with the generation of different zones of proliferative (periphery) to slow cycling, quiescent and apoptotic cells (inner core), while circumventing the cost and complexity of animal studies.

IVK8/Gelatin hybrid hydrogels that were used for the culture of HepG2 (FIG. 4) and SW480 (FIG. 5) tumor spheroids resulted in similar growth and viability as those cultured using Geltrex (P>0.05). Notably, the growth of the tumor spheroids hydrogels prepared using IVK8 solely was significantly impaired, thereby illustrating the importance of incorporating gelatin into the matrix for supporting cell growth.

To confirm whether the spheroid cultures exhibit cellular activities that are typical of those grown in Geltrex, live/dead staining, hypoxia detection, as well as immunohistochemical staining of Ki67 and Cleaved Caspase 3 were performed. Live/dead staining shows that there are more viable cells than dead/dying cells at the spheroid periphery while more dead/dying cells are present in the core. This observation is similar for both the IVK8/Gelatin hydrogels and Geltrex. From FIG. 6B, it was observed that hypoxia occur at the core of the spheroids and this do not differ between Geltrex, IVK8/Gelatin hybrid hydrogels as well as IVK8 peptide hydrogel. The treatment of tumor spheroids cultured in Geltrex and IVK8/Gelatin hybrid hydrogels with Dox showed similar decreasing trend in size measurements (FIG. 6C), thereby showing that the hybrid hydrogels can be used as a scaffold for in vitro drug screening studies. As for the detection of proliferation (Ki67) and apoptosis (Cleaved Caspase 3) markers, FIG. 6D shows that Ki67 is mainly found at the periphery of the spheroids while most of the Cleaved Caspase 3 is detected at the core of the spheroids. The proliferative and apoptotic regions appear similar between Geltrex, IVK8/Gelatin hybrid hydrogels and IVK8 peptide hydrogel.

### In Vivo Expansion of Tumors With IVK8/Gelatin Hybrid Hydrogels as Supporting Matrix

Basement membrane products (e.g. Matrigel and Geltrex) have been widely used as matrices to support tumor cell growth in animal models. With the aim to provide synthetic alternatives to such commercial matrices, it is important to test the practicality and performance of the IVK8/Gelatin hydrogels to provide a comparison with these products for in vivo applications. In both studies conducted, the tumor growth is similar between IVK8/Gelatin hybrid hydrogels and Geltrex as both tumor volume and weight measurements showed no significant difference (P>0.05). It is also important to note that for hydrogel prepared with IVK8 solely I_0.33%, the tumor cells did not form solid tumors, and again demonstrates the importance of incorporating gelatin into the hybrid hydrogels for promoting cell growth, this time for in vivo application. Furthermore, the hydrogels did not cause any body weight loss in the animals, thereby showing that the IVK8/gelatin hybrid hydrogels is biocompatible for in vivo usage.

In summary, a peptide and gelatin hybrid hydrogel has been developed as a potential alternative to animal-derived basement membrane products for cell culture applications. The hybrid hydrogel showed equivalent performance with regards to cell proliferation, spheroid characteristics, biocompatiblity as well as in vivo tumor growth when compared against Geltrex. Furthermore, the simplicity of its material composition, absence of xenogenic components and low cost of production confers prodigious incentives to utilize this hydrogel for both in vitro and in vivo cell/tissue culture.

The peptide/gelatin hybrid hydrogels provided similar growth and viability of the cancer cells when they were used as scaffolds for in vitro 3D culture of tumor spheroids. Hypoxic, proliferative and apoptotic characteristics of the tumor spheroids were also similar between the peptide/gelatin hybrid hydrogels and Geltrex. In vivo expansion of solid tumors was similar between peptide/gelatin hybrid hydrogels and Geltrex in mice model. In all, the simplicity of its material composition, absence of xenogenic components confers prodigious incentives to utilize this hydrogel for both in vitro and in vivo cell/tissue culture.

The chemical composition of peptide/gelatin hybrid hydrogels is well-defined and does not contain animal components, unlike common basement membrane products (e.g. Matrigel). The performance in cell expansion of peptide/gelatin hybrid hydrogels is equivalent of commercial product, Geltrex (Thermofisher).

The hybrid hydrogels described herein may be used for 3D cell culture, stem cell culture, in vitro tumor spheroids generation, and in vivo solid tumor development.

1. A hydrogel comprising a peptide and gelatin, the peptide comprising a sequence having at least 8 amino acids with alternating hydrophobic amino acids (X) and hydrophilic amino acids (Y), wherein each hydrophobic amino acid is independently selected from the group consisting of isoleucine (I), valine (V) and leucine (L), and each hydrophilic amino acid is independently selected from the group consisting of arginine (R), lysine (K), glutamic acid (E), and aspartic acid (D), wherein the hydrophilic amino acids are selected such that there is at least one arginine and at least one lysine.

2. The hydrogel according to claim 1, wherein the hydrophilic amino acids are selected such that there are at least two hydrophilic amino acids selected from the group consisting of glutamic acid and aspartic acid.

3. The hydrogel according to claim 1, wherein the peptide comprises 8 or 12 amino acids.

4. The hydrogel according to claim 1, wherein four sequential hydrophilic amino acids (Y₁, Y₂, Y₃, and Y₄) in the sequence are selected such that Y₁ and Y₂ are each independently selected from the group consisting of glutamic acid and aspartic acid, and Y₃ and Y₄ are each independently selected from the group consisting of arginine and lysine.

5. The hydrogel according to claim 4, wherein Y₁ and Y₂ is are glutamic acid, Y₃ is arginine, and Y₄ is lysine.

6. The hydrogel according to claim 1, wherein six sequential hydrophilic amino acids (Y₅, Y₆, Y₁, Y₂, Y₃, and Y₄) in the sequence are selected such that Y₁ and Y₂ are each independently selected from the group consisting of glutamic acid and aspartic acid, and Y₅, Y₆, Y₃ and Y₄ are each independently selected from the group consisting of arginine and lysine.

7. The hydrogel according to claim 6, wherein Y₁ and Y₂ are glutamic acid, Y₅ and Y₃ are arginine, and Y₆ and Y₄ are lysine.

8. The hydrogel according to claim 1, wherein the sequence comprises a SEQ ID selected from the group consisting of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16.

9. The hydrogel according to claim 1, wherein the peptide is present in 0.1 to 2 weight percent of the hydrogel.

10. The hydrogel according to claim 9, wherein the peptide is present in 0.2 to 1 weight percent of the hydrogel.

11. The hydrogel according to claim 1, wherein gelatin is present in 1 to 10 weight percent of the hydrogel.

12. The hydrogel according to claim 11, wherein gelatin is present in 1 to 6 weight percent of the hydrogel.

13. A method of preparing a hydrogel, the method comprising mixing a peptide and gelatin under suitable conditions to form the hydrogel, the peptide comprising a sequence having at least 8 amino acids with alternating hydrophobic amino acids and hydrophilic amino acids, wherein each hydrophobic amino acid is independently selected from the group consisting of isoleucine (I), valine (V) and leucine (L), and each hydrophilic amino acid is independently selected from the group consisting of arginine (R), lysine (K), glutamic acid (E), and aspartic acid (D), wherein the hydrophilic amino acids are selected such that there is at least one arginine and at least one lysine.

14. The method according to claim 13, wherein the suitable conditions include at least one of the following conditions: (a) a salt; and (b) a temperature of 20° C. to 40° C.

15. A kit for preparing a hydrogel, the kit comprising a peptide and gelatin, the peptide comprising a sequence having at least 8 amino acids with alternating hydrophobic amino acids and hydrophilic amino acids, wherein each hydrophobic amino acid is independently selected from the group consisting of isoleucine (I), valine (V) and leucine (L), and each hydrophilic amino acid is independently selected from the group consisting of arginine (R), lysine (K), glutamic acid (E), and aspartic acid (D), wherein the hydrophilic amino acids are selected such that there is at least one arginine and at least one lysine.

16. A method of culturing cells, the method comprising
providing the hydrogel according to claim 1; and
incubating a cell and the hydrogel under suitable conditions to culture the cell.

17. The method according to claim 16, wherein the cell is a tumor cell.

18. The method according to claim 16, wherein the cell is a stem cell.

19. A method of determining an effect of one or more compounds on a plurality of cells, the method comprising
culturing the plurality of cells according to claim 16;
adding the one or more compounds to the plurality of cells; and
determining the effect of the one or more compounds on the plurality of cells.
